(19) United States
(12) Patent Application Publication
(10) Pub. No.: US 2003/0073969 A1
(43) Pub. Date: Apr. 17, 2003

(54) Surgical sponge and method for making the same

(76) Inventor: Peter S. Klainer, Sterling, VA (US)

Correspondence Address: ROTHWELL, FIGG, ERNST & MANBECK, P.C., 1425 K STREET, N.W., SUITE 800, WASHINGTON, DC, 20005, US

(21) Appl. No.: 09/978,618

(22) Filed: Oct. 17, 2001

(51) Int. Cl. A61F 13/15

(52) U.S. Cl. 604/385.01

(57) ABSTRACT

A surgical sponge comprising a center portion which, in one embodiment, is hexagonal shaped, and five or more extending portions which, in one embodiment, are six triangle-shaped portions, and are coupled to the center portion to form a star-shaped surgical sponge. The surgical sponge of the present invention may also include a radio-opaque marker attached to the center portion or, alternatively, radio-opaque material may be incorporated in the material used to form the sponge. The surgical sponge of the present invention is economical, efficient for use in blotting, and suitable for compact folding and packaging.

### COPYRIGHT NOTIFICATION

Portions of this patent application contain materials that is subject to copyright protection. The copyright owner has no objection to the facsimile reproduction by anyone of the patent document, or the patent disclosure, as it appears in the Patent and Trademark Office.

### BACKGROUND OF THE INVENTION

1. Field of the Invention

The present invention relates, generally, to absorbent articles and, more particularly, to surgical sponges or pads.

2. Discussion of the Background

During surgical procedures, body fluids are often released into the surgical cavity of the patient. Surgical sponges, such as for example a laparotomy sponge or pad, are commonly used during surgical procedures to absorb the body fluids of the patient (e.g., blood, serum, plasma, lymph, spinal fluid, tissue fluid, urine, sweat, bile fluid, digestive fluids, etc.) both inside and near the site of the surgery. It is common practice to blot the incision and the surrounding areas with sponges to remove the body fluids from the patient.

Surgical sponges, such as laparotomy pads, have served as the basic absorbent article used in medical applications, particularly surgical operations, for absorbing body fluids, especially blood. Laparotomy sponges, for example, most commonly comprise a plurality of layers of absorbent loosely-woven gauze which are bound to one another along the side edges of the overlying layers of gauze. The general theory behind the use of multilayered gauze in laparotomy sponges is that the openness of the weave of the gauze, plus the bulk provided by the multiple layers, permits the sponge to rapidly take up substantial volumes of body fluids.

Conventional surgical sponges, such as Lap Sponges available from Medical Action Industries, Inc. of Asheville, N.C., are normally lint-free, gauze cloth squares or, in other cases, are rectangular in shape. Because the blotting action performed by the surgeon or attending surgical personnel often occurs in small, difficult to reach, and/or focused areas, often the comers of the sponge are used. In fact, in most cases, the comers of the surgical sponge are the only portions used for blotting, since the comers permit easier blotting of smaller spaces, hard to reach locations, and/or focused areas within the surgical opening.

As is well recognized in the art, a surgical sponge, such as a laparotomy sponge, when soaked with blood, very strikingly resembles normal body tissue found within a patient's body cavity. As a consequence, a strict accounting for all implements and surgical supplies used during surgical procedures is required, especially with respect to laparotomy sponges (and other sponge types). As a means to ensure detection in the event that a surgical sponge used during a surgical procedure is unaccounted for, such sponges are typically provided with at least one radio-opaque element, which is in some manner incorporated into the structure of the sponge to ensure its integrity with the sponge during use of the sponge and thereafter. This radio-opaque element is intended to be readily detectable under x-ray examination. If, after completion of a surgical procedure, all sponges cannot be accounted for, the patient is examined by x-ray equipment in an attempt to ensure that a “missing” sponge has not been left in the patient's body cavity.

The radio-opaque marker, such as barium sulfate dispersed in a suitable carrier, is normally attached to a comer of the sponge to assist in locating the sponge by x-ray should it be inadvertently left in the patient's body after surgery. As a consequence of the radio-opaque marker being attached to a comer of the sponge, the comer with the attached strip is typically not used to blot fluids. Consequently, conventional square and rectangular sponges having four comers provide only three comers that are suitable for blotting.

In most cases, the comers of the sponge are the only regions of the sponge used prior to discarding the sponge. After discarding the sponge, the surgeon, nurse or other operating room personnel must be provided with a new sponge for use during the procedure. Consequently, each time a sponge is discarded, the blotting (and possibly other surgical activity) is interrupted for a time until a new sponge is made available for blotting. Thus, the more often the sponge must be replaced, the greater the frequency of interruptions and associated delays in the surgical procedure, which in turn may impact the total length of the surgical procedure. Further, the configuration of the conventional square or rectangular surgical sponge with its limited number of comers per sponge results in the use of an increased number of sponges during a given surgical procedure and an inability to utilize a significant portion of the overall surface area of the sponge.

Thus, notwithstanding the available surgical sponges, there is a need for a surgical sponge: (1) that can be used for a longer period of time during a surgical procedure; (2) that can be used to blot more than the limited number of times blotting is possible with the available three or four comers of a conventional surgical sponge; (3) that has available for use to perform blotting a significant portion of the total surface area of the surgical sponge; and (4) that can substantially reduce the total number of surgical sponges used during a surgical procedure.

### SUMMARY OF THE INVENTION

The primary object of the present invention is to overcome the deficiencies of the prior art surgical sponges described above by providing a sponge with a greater number of blotting portions or areas.

Another key object of the present invention is to provide a surgical sponge that can be used for a longer period of time during a surgical procedure.

Still another key object of the present invention is to provide a surgical sponge that can be used to blot more than the limited number of times blotting is possible with the available three or four comers of a conventional surgical sponge.

Yet another key object of the present invention is to provide a surgical sponge that has available for use to perform blotting a significant portion of the total surface area of the surgical sponge.

Another key object of the present invention is to provide a surgical sponge that can substantially reduce the total number of surgical sponges used during a surgical procedure.

Yet another object of the present invention is to provide a sponge that is easy to use.

Still another object of the present invention is to provide a sponge that is more economical than prior art sponges.

Another object of the present invention is to provide a sponge that is easy to manufacture and package.

The present invention achieves these objects and others by providing a surgical sponge comprising a center portion which, in one embodiment, is hexagonal shaped, and five or more extending portions which, in one embodiment, are six triangle shaped portions, and are attached to the center portion to form a star-shaped surgical sponge. The surgical sponge of the present invention may also include a radio-opaque marker attached to the center portion or, alternatively, radio-opaque material may be incorporated in the material used to form the sponge. The surgical sponge of the present invention is economical, efficient for use in blotting, and suitable for compact folding and packaging.

In a second embodiment of the present invention, a surgical sponge is provided comprising a circular-shaped center portion and eight (or more) triangular-shaped, spaced-apart extending portions integral on one side with the center portion. A radio-opaque marker may be attached to the center portion of the sponge or radio-opaque material may be incorporated in the material used to form the sponge.

In another embodiment, a surgical sponge comprising a circular shaped center portion and eight (or more) spaced-apart extending portions in a saw-tooth configuration is provided.

Further features and advantages of the present invention, as well as the structure and operation of various embodiments of the present invention, are described in detail below with reference to the accompanying drawings.

### BRIEF DESCRIPTION OF THE DRAWINGS

The accompanying drawings, which are incorporated herein and form part of the specification, illustrate various embodiments of the present invention and, together with the description, further serve to explain the principles of the invention and to enable a person skilled in the pertinent art to make and use the invention. In the drawings, like reference numbers indicate identical or functionally similar elements.

A more complete appreciation of the invention and many of the attendant advantages thereof will be readily obtained as the same becomes better understood by reference to the following detailed description when considered in connection with the accompanying drawings, wherein:

FIG. 1 is a planar view of a surgical sponge of the present invention.

FIG. 2 is a representation of one embodiment of the surgical sponge of the present invention including a radio-opaque marker.

FIG. 3 is a representation of one embodiment of the surgical sponge of the present invention depicting the fold lines for folding of the sponge for packaging.

FIGS. 4A-B are planar views of a second embodiment of the surgical sponge of the present invention in the folded and unfolded positions, respectively.

FIGS. 5A-B are planar views of a third embodiment of the surgical sponge of the present invention in the folded and unfolded positions, respectively.

### DETAILED DESCRIPTION OF THE PREFERRED EMBODIMENTS

In the following description, for purposes of explanation and not limitation, specific details are set forth, such as sponge material, size, manufacturing methods, methods of attaching a radio-opaque marker, etc. in order to provide a thorough understanding of the present invention. However, it will be apparent to one skilled in the art that the present invention may be practiced in other embodiments that depart from these specific details. Detailed descriptions of well-known sponge material, methods of attaching or incorporating a radio-opaque marker, manufacturing methods, and methods of packaging are omitted so as not to obscure the description of the present invention. In addition, as will be evident to those skilled in the art, the present invention could be used in place of, or in conjunction with, the currently available surgical pads and sponges, including laparotomy pads.

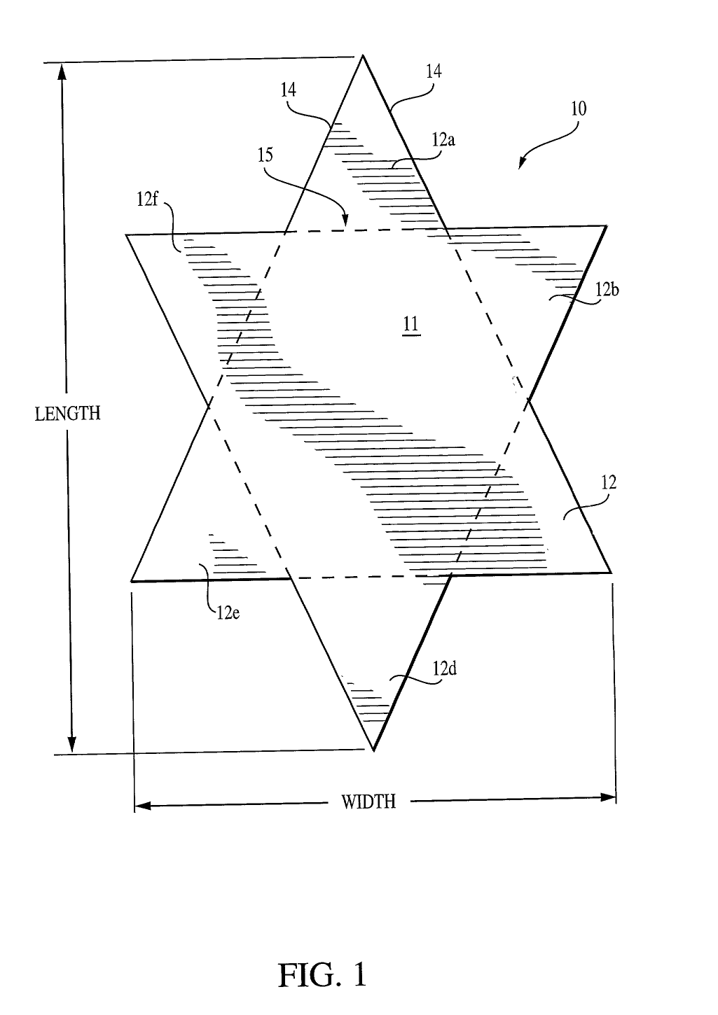

In one embodiment of the present invention, the sponge 10 is configured to include five or more extending portions that are suitable for blotting. As shown in FIG. 1, the sponge 10 is shaped as a six-point star. In a first sample of the star-shaped surgical sponge, the dimensions are: a length of eighteen (18) inches and a width of twelve (12) inches. The sponge is comprised of a center portion 11 and six extending portions 12a-f that extend from the center portion 11. (The distinction between center portion 11 and extending portions 12a-f is indicated by the dotted lines in FIG. 1.) In this example embodiment, the center portion 11 is hexagonal in shape and the sponge is constructed of lint free, cotton gauze, although any suitable material may be used. The extending portions 12a-f are comprised of two external sides 14 that are straight and an integral side 15 (also indicated by the dotted lines in FIG. 1) that is straight. In the present embodiment, each of the extending portions 12a-f are spaced-apart and, thus, separate from every other extending portion 12, and are integral only with the center portion 11. In addition, the angle at which the two external sides 14 of each extending portion 12a-f meet is less than ninety degrees (90°).

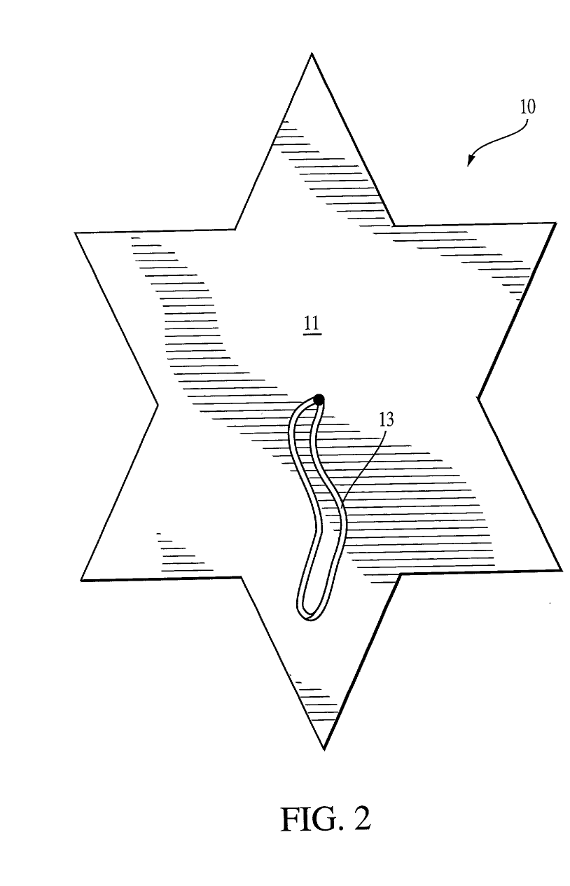

As shown in FIG. 2, a radio-opaque marker 13 is attached to the center portion 11 of the sponge 10 in a conventional manner. The radio-opaque marker 13 in the present embodiment is a radio-opaque strip. By attaching the marker 13 to the center portion 11 of the sponge 10, instead of to an extending portion 12 of the sponge 10, all of the extending portions 12 of the sponge 10 may be used for blotting. Thus, where the prior art sponge with four corners had three portions suitable for blotting, the present example embodiment has six. The radio-opaque marker 13 may be attached to the sponge in any location on the center portion. Alternatively, a radio-opaque material may be incorporated into the material used to form the surgical sponge.

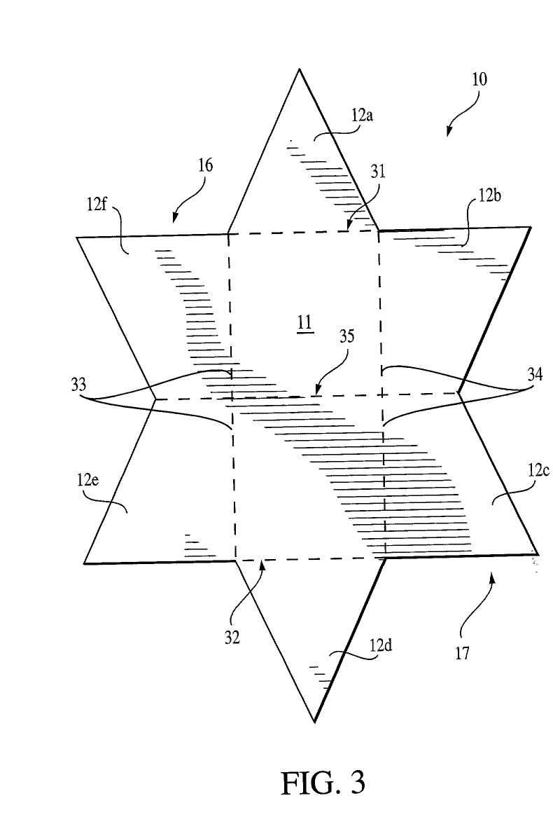

The example embodiment of the present invention is also easily folded for packaging according to the folding lines of FIG. 3. The first step in folding the sponge is to fold extending portion 12a towards center portion 11 along fold line 31, and to fold extending portion 12d towards center portion 11 along fold line 32. The next step in folding the sponge includes folding the left portion 16 of the sponge 10 toward the center of the sponge along fold line 33. Next, the right portion 17 of the sponge 10 is folded toward the center of the sponge along fold line 34 to provide a substantially rectangular shape (shaped according to fold lines 31-34). Finally, the rectangular shaped partially folded sponge is folded in half along fold line 35 into a square shape.

The sponge can be of any desired thickness, and preferably in the range of about 1.0 to about 3.0 millimeters in thickness, which is sufficient for fluid absorption, possesses adequate permeability for use with fluid suction devices, and is sufficiently pliable to serve as a tissue wipe.

In the present embodiment, the sponge is constructed from an absorbent material (cotton) comprising a plurality of layers of open mesh gauze. As is well-known in the art, the gauze is preferably woven in a weave pattern suitable for use both as a drape in a surgical procedure and in the nature of a laparotomy sponge

The layers of the soft, low-count gauze like material are typically unified along fairly widely separated lines usually extending longitudinally or transversely, but in some instances in both directions (as in the first example embodiment). Unification may be accomplished either by lines of machine stitching, when the pack is formed of separated layers or, as is more common, by folding a single width of fabric, or by interweaving yarns in various layers of the multi-ply fabric. Any suitable method of unification may be used.

Alternately, the sponge could be constructed of a nonwoven material. The nonwoven fabric may comprise any suitable combination of natural and/or synthetic textile materials including cotton, rayon, acrylics, polyester and nylon.

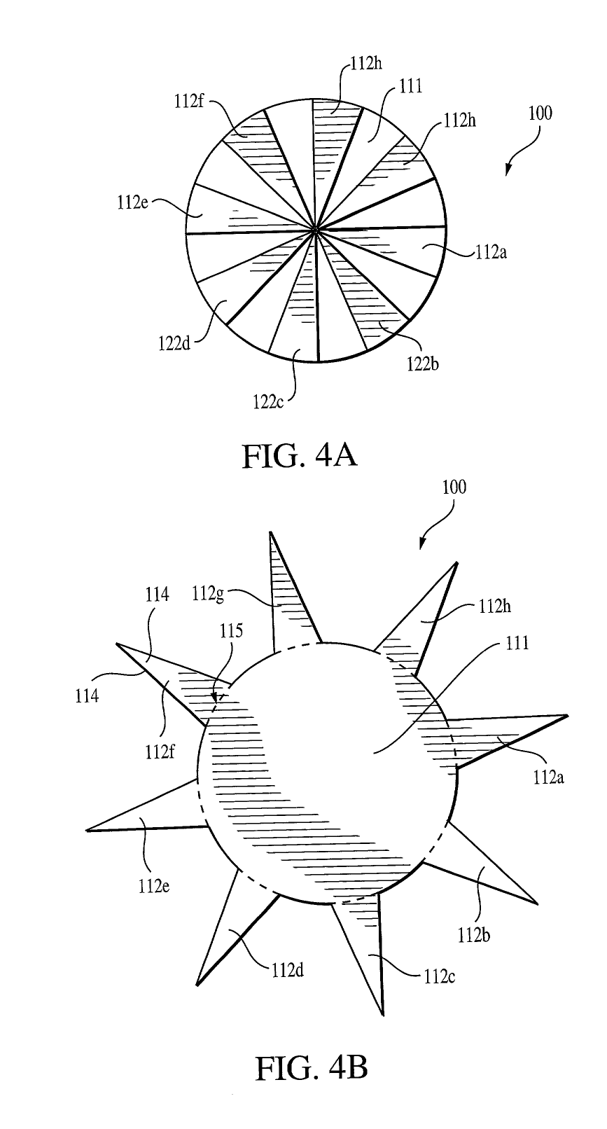

Referring to FIGS. 4A-B, a second embodiment of the present invention is shown. In the second embodiment of the surgical sponge of the present invention, the sponge 100 is configured with eight-points. The sponge 100 is comprised of a center portion 111 and spaced-apart extending portions 112a-h. The radius of the center portion 111 may be in the range of 2-4inches, for example. Each of the extending portions is substantially triangular in shape and extends 2-6 inches from the center portion, for example. The eight extending portions 112a-h extend from the center portion 111. (The distinction between center portion 111 and extending portions 112a-h are indicated by the dotted lines in FIG. 4B.) In this example embodiment, the center portion 111 is circular in shape and the sponge is constructed of lint free, cotton gauze, although any suitable material may be used. The extending portions 112a-h are comprised of two external sides 114 that are straight and an integral side 115 (also indicated by the dotted lines in FIG. 4B) that is curved. In the present embodiment, each of the extending portions 112a-h are spaced-apart and, thus separate from every other extending portion 112, and are integral only with the center portion 111. In addition, the angle at which the two external sides 114 of each extending portion 112a-h meet is less than ninety degrees (90°). The radio-opaque marker is not shown, but may be attached at any suitable place on the sponge 100, and preferably at the center of the center portion 111. Alternatively, radio-opaque material may be incorporated into the material used to form the sponge.

The example embodiment shown in FIG. 4A is in its folded configuration, in which each extending portion 112a-h is folded along its integral side 115 toward the center of the center portion 111.

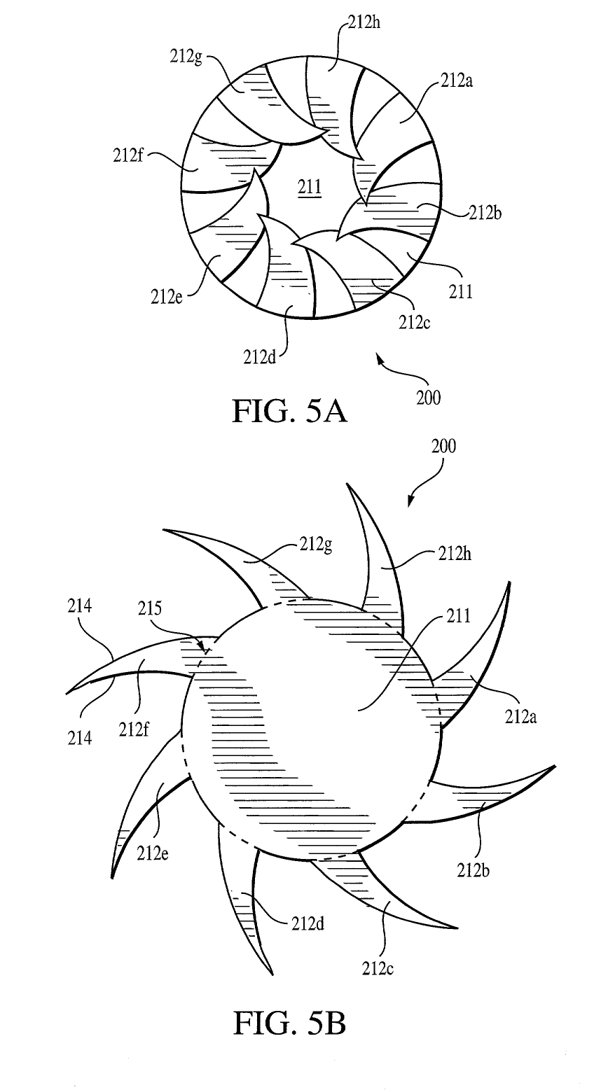

Referring to FIGS. 5A-B, a third embodiment of the present invention is shown. In the third embodiment of the surgical sponge of the present invention, the sponge 200 is configured to have eight-points with a saw-tooth shaped configuration. The sponge 200 is comprised of a center portion 211 and eight spaced-apart extending portions 212a-h that extend from the center portion 211. (The distinction between center portion 211 and extending portions 212a-h are indicated by the dotted lines in FIG. 5B.) The radius of the center portion 211 may be in the range of 2-4 inches, for example. Each of the extending portions 212a-h is substantially triangular in shape and extends 2-6 inches from the center portion, for example. The center portion 211 is circular in shape and the sponge is constructed of lint free, cotton gauze, although any suitable material may be used. The extending portions 212a-h are comprised of two external sides 214 that are curved and an integral side 215 (also indicated by the dotted lines in FIG. 5B) that is curved. In the present embodiment, each of the extending portions 212a-h spaced-apart and, thus, are separate from every other extending portion 212, and are integral only with the center portion 211. In addition, the angle at which the two external sides 214 of each extending portion 212a-h meet is less than ninety degrees (90°). The radio-opaque marker is not shown, but may be attached at any suitable place and, preferably, at the center of the center portion 211. Alternatively, radio-opaque material may be incorporated into the material used to form the sponge.

The example embodiment is shown in FIG. 5a in its folded configuration in which each extending portion 212a-h is folded along its integral side 115 toward the center of the center portion 211.

While the example embodiments described herein employ six and eight extending portions 12, respectively, other embodiments may use five extending portions, seven extending portions, or more than eight extending portions, depending on the intended use. While in the example embodiments described herein, the center portion is hexagonal in shape or circular in shape, other configurations may employ a center portion having a different shape (e.g., rectangular, pentagon, octagon, square, or oval). In addition, instead of employing two external sides 14 that are straight, the external sides could be curved so that the extending portion is rounded on its end (as opposed to being pointed as in the example embodiments). While in the above example embodiments, the angle at which the two external sides 14 of each extending portion 12 meet is less than ninety degrees, the angle could be greater than ninety (90°) degrees or much less than ninety degrees (90°) (e.g., seventy-five degrees (75°), sixty degrees (60°), or less) in alternative embodiments of the present invention. Finally, while the extending portions 12 of the above example are the same or slightly different in size and in shape, other embodiments might employ extending portions that are similar in size and shape, or that are different in size and/or shape.

The foregoing has described the principals, embodiments, and modes of operation of the present invention. However, the invention should not be construed as being limited to the particular embodiments described above, as they should be regarded as being illustrative and not as restrictive. It should be appreciated that variations may be made in those embodiments by those skilled in the art without departing from the scope of the present invention.

While a preferred embodiment of the present invention has been described above, it should be understood that it has been presented by way of example only, and not limitation. Thus, the breadth and scope of the present invention should not be limited by the above described exemplary embodiments.

Obviously, numerous modifications and variations of the present invention are possible in light of the above teachings. It is therefore to be understood that within the scope of the appended claims, the invention may be practiced otherwise than as specifically described herein.

What is claimed is:

1. A surgical sponge made of an absorbent material comprising:
a center portion having six connecting sides; and
six extending portions connected to said center portion,
wherein each of said extending portions includes a first side, a second side, and a third side, wherein said first side is connected to the connecting side of the center portion; said second side is connected on a first end to said first side and on a second end to said third side, and said third side is connected on a first end to said first side and on a second end to said second side.

2. The surgical sponge of claim 1, further comprising a radio-opaque marker attached to said center portion.

3. The surgical sponge of claim 1, further comprising a radio-opaque material, wherein said radio-opaque material is incorporated into the absorbent material.

4. The surgical sponge of claim 1, wherein said center portion is hexagonal-shaped.

5. The surgical sponge of claim 1, wherein each of said extending portions is triangle-shaped.

6. The surgical sponge of claim 1, wherein the angle which is formed at the connection of the second end of the second side and the second end of the third side is less than ninety degrees.

7. A surgical sponge comprising:
a center portion; and
at least five extending portions attached to said center portion, each of said extending portions including a first end, which is attached to said center portion, and a second end, which extends away from said center portion and is spaced-apart from the second end of every other extending portion.

8. The surgical sponge of claim 7, further comprising a radio-opaque marker.

9. The surgical sponge of claim 8, wherein said marker is attached to said center portion.

10. The surgical sponge of claim 7, wherein said sponge is made of an absorbent material and further comprises a radio-opaque material, wherein said radio-opaque material is incorporated into the absorbent material.

11. The surgical sponge of claim 7, wherein at least one of said extending portions includes first and second substantially straight external sides.

12. The surgical sponge of claim 11, wherein said first and second substantially straight external sides are joined together at said second end and form an angle that is less than ninety degrees.

13. The surgical sponge of claim 7, wherein at least one of said extending portions does not include a substantially straight external side.

14. The surgical sponge of claim 7, wherein said at least five extending portions includes six extending portions.

15. The surgical sponge of claim 7, wherein said at least five extending portions includes eight extending portions.

16. The surgical sponge of claim 7, wherein said center portion includes at least five substantially straight connecting sides coupled to said at least five extending portions.

17. The surgical sponge of claim 7, wherein:
said center portion includes six substantially straight connecting sides;
said at least five said extending portions includes six extending portions; and
said six substantially straight connecting sides of said center portion are coupled to said six extending portions.

18. The surgical sponge of claim 7, wherein said sponge is a laparotomy pad.

19. A method of manufacturing an absorbent surgical sponge constructed of a plurality of layers of absorbent woven material, comprising the steps of:
providing a plurality of layers of absorbent woven material;
forming a center portion; and
forming at least five extending portions coupled to said center portion.

20. The method of claim 19, further comprising the step of:
coupling a radio-opaque marker to said center portion.
